(19) United States
(12) Patent Application Publication
(10) Pub. No.: US 2013/0304143 A1
(43) Pub. Date: Nov. 14, 2013

(54) MEDICAL DATA STORAGE AND COMMUNICATION SYSTEM

(71) Applicant: Isabelle Banville, Newcastle, WA (US)

(72) Inventor: Isabelle Banville, Newcastle, WA (US)

(73) Assignee: PHYSIO-CONTROL, INC., Redmond, WA (US)

(21) Appl. No.: 13/890,236

(22) Filed: May 8, 2013

Related U.S. Application Data
(60) Provisional application No. 61/644,209, filed on May 8, 2012.

(51) Int. Cl. A61N 1/39

(52) U.S. Cl. CPC A61N 1/3993
USPC 607/5; 607/60

(57) ABSTRACT

An external medical device can include a medical data collection port for collecting medical data corresponding to a person using the external medical device, a radio frequency identification (RFID) communication module, and a processor configured to cause the RFID communication module to provide the medical data to an RFID device that is external to the external medical device.

### RELATIONSHIP WITH OTHER APPLICATIONS

The present patent application claims the benefit of U.S. Provisional Patent Application Ser. No. 61/644,209, filed on May 8, 2012, the disclosure of which is hereby incorporated by reference for all purposes.

### FIELD

The present subject matter generally relates to the field of medical devices such as defibrillators.

### BACKGROUND

In humans, the heart beats to sustain life. In normal operation, the heart propels blood through the various parts of the body. The chambers of the heart contract and expand in a periodic, regular, and coordinated fashion. The sequence is as follows; The right atrium sends deoxygenated blood into the right ventricle. The right ventricle pumps the blood to the lungs, where, before returning to the left atrium, the blood becomes oxygenated. The left atrium pumps the oxygenated blood to the left ventricle. The left ventricle then expels and forces the blood to circulate through the various parts of the body.

The heart chambers pump because of the heart's electrical control system. More particularly, the sinoatrial (SA) node generates an electrical impulse, which cascades into electrical signals. The electrical signals in turn cause the above-described contractions of the various chambers in the heart in the correct sequence. The electrical pattern created by the sinoatrial (SA) node is called a sinus rhythm.

Unfortunately, sometimes the electrical control system of the heart malfunctions and causes the heart to beat irregularly, or not at all. The cardiac rhythm is then generally called an arrhythmia. Arrhythmias may be caused by electrical activity from locations in the heart other than the SA node. Some types of arrhythmia may result in inadequate blood flow, thus reducing the amount of blood pumped to the rest of the body. Some arrhythmias may even result in a Sudden Cardiac Arrest (SCA). In a SCA, the heart fails to pump blood effectively, and if not treated, death can occur. It is estimated that SCA results in more than 250,000 deaths per year in the United States alone. Further, a SCA may result from a condition other than an arrhythmia.

One type of arrhythmia associated with SCA is known as Ventricular Fibrillation (VF). VF is a type of malfunction where the ventricles make rapid, chaotic, spasm-like movements, instead of the normal, coordinated, sequential rhythmic contractions. When arrhythmia happens, the heart does not pump enough blood to deliver enough oxygen to the vital organs. The person's condition deteriorates rapidly and, if not reversed, the person may expire within minutes.

Ventricular Fibrillation can often be reversed using a life-saving device called a defibrillator. A defibrillator, when applied properly and promptly, can administer an electrical shock to the heart and terminate the VF, giving the heart an opportunity to resume proper functioning. If the VF is not terminated with the initial shock, subsequent shock may be administered, often at escalating energies.

A challenge with defibrillation is that the electrical shock, if not immediately at the onset, must be administered as soon as possible right after the onset of the VF. There is not much time. The survival rate of persons suffering from VF decreases by about 10% for each minute the administration of a defibrillation shock is delayed. After about 10 minutes, the rate of survival for SCA victims averages less than 2%.

During VF, a person's condition deteriorates rapidly because the blood is not flowing to the brain, heart, lungs, and other organs. If resuscitation attempts are to be successful and damage to organs prevented, blood flow must be restored.

To-date, the challenge of administering a shock and defibrillating as quickly as possible and within minutes of the onset of VF has been approached in a number of ways. Great efforts and training are constantly being implemented to ensure as short of an emergency teams response time as possible. Great efforts and education are being implemented in communities to empower lay bystanders to respond to such events as quickly and efficiently as possible.

Cardiopulmonary Resuscitation (CPR) is one method of forcing blood flow in a person experiencing cardiac arrest. In addition, CPR is the primary recommended treatment for some patients with some kinds of non-VF cardiac arrest, such as asystole and pulseless electrical activity (PEA). CPR is a combination of techniques that include chest compressions to force blood circulation, and rescue breathing to force respiration.

Properly administered CPR provides oxygenated blood to critical organs of a person in cardiac arrest, thereby minimizing the deterioration that would otherwise occur. CPR can be beneficial for persons experiencing VF because it slows the deterioration that would otherwise occur while a defibrillator is being retrieved. Indeed, for patients with an extended down-time, survival rates are higher if CPR is administered prior to defibrillation. Advanced medical devices often can also coach a rescuer who performs CPR. For example, a medical device can issue instructions, and even prompts, for the rescuer to perform CPR more effectively. VF can occur unpredictably, even to a person who is not considered at a high risk and cardiac events can be experienced by people who lack the benefit of an Implantable Cardioverter Defibrillator (ICD) therapy.

For people who are considered to be at a higher risk of VF or other heart arrhythmias, when indicated, an ICD can be implanted surgically. An ICD can monitor the person's heart, and administer an electrical shock promptly. An ICD reduces the need to have the higher-risk person be monitored constantly by medical personnel.

When VF occurs to a person who does not have an ICD, they collapse, because blood flow has stopped. They should receive therapy quickly. For a VF victim without an ICD, a different type of defibrillator can be used, which is called an external defibrillator. External defibrillators have been made portable, so they can be brought by a bystander/rescuer to a potential VF victim quickly enough to revive them. The patient's life may hinge on the bystander/rescuer quick and efficient response to the situation. The time from the collapse to the time a portable defibrillator is applied to the cardiac event victim is critical.

### BRIEF SUMMARY

The present description gives instances of medical devices, software and methods, the use of which may help overcome problems and limitations of the prior art.

In some embodiments, an external medical device can include a housing, a medical data collection port for collecting medical data corresponding to a person using the external medical device, a radio frequency identification (RFID) communication module in the housing, and a processor in the housing configured to cause the RFID communication module to transmit the medical data to an RFID device that is external to the external medical device.

In other embodiments, an external medical device can include a housing, a medical data collection port for collecting medical data corresponding to a person using the external medical device, a communication module in the housing, and a processor in the housing configured to cause the communication module to transmit the medical data to a memory device integrated with a removable battery.

An advantage over the prior art is that patient medical data may be transmitted quickly, easily, and securely from an external medical device, e.g., an external defibrillator, to an external memory device, e.g., an RFID tag or a flash memory device. The medical data may then be transmitted from the external memory device to a remote service, for example, by way of a network, e.g., such as the Internet. Such data transfer may be performed independent of a user needing to physically establish a connection between the devices by plugging the memory device into the external medical device, for example.

These and other features and advantages of this description will become more readily apparent from the following Detailed Description, which proceeds with reference to the drawings, in which:

### BRIEF DESCRIPTION OF THE DRAWINGS

FIG. 1 is a diagram of a scene where an external defibrillator is used to save the life of a person according to embodiments.

FIG. 2 is a table listing different types of the external defibrillator shown in FIG. 1, and who they might be used by.

FIG. 3 is a functional block diagram showing components of an external defibrillator, such as the one shown in FIG. 1, which is made according to embodiments.

FIG. 4 is a diagram showing an external defibrillator transmitting medical data to an RFID device according to embodiments.

FIG. 5 is a diagram showing the RFID device of FIG. 4 providing at least some of the medical data to a base station according to embodiments.

FIG. 6 is a diagram showing an external medical device transmitting medical data to a memory device in a removable battery according to embodiments.

FIG. 7 is a diagram showing the memory device of FIG. 6 providing at least some of the medical data to a battery charging and data transfer device according to embodiments.

FIG. 8 is a flowchart for illustrating example methods executable by external defibrillators according to embodiments.

FIG. 9 is a flowchart for illustrating example methods executable by external medical devices according to embodiments.

### DETAILED DESCRIPTION

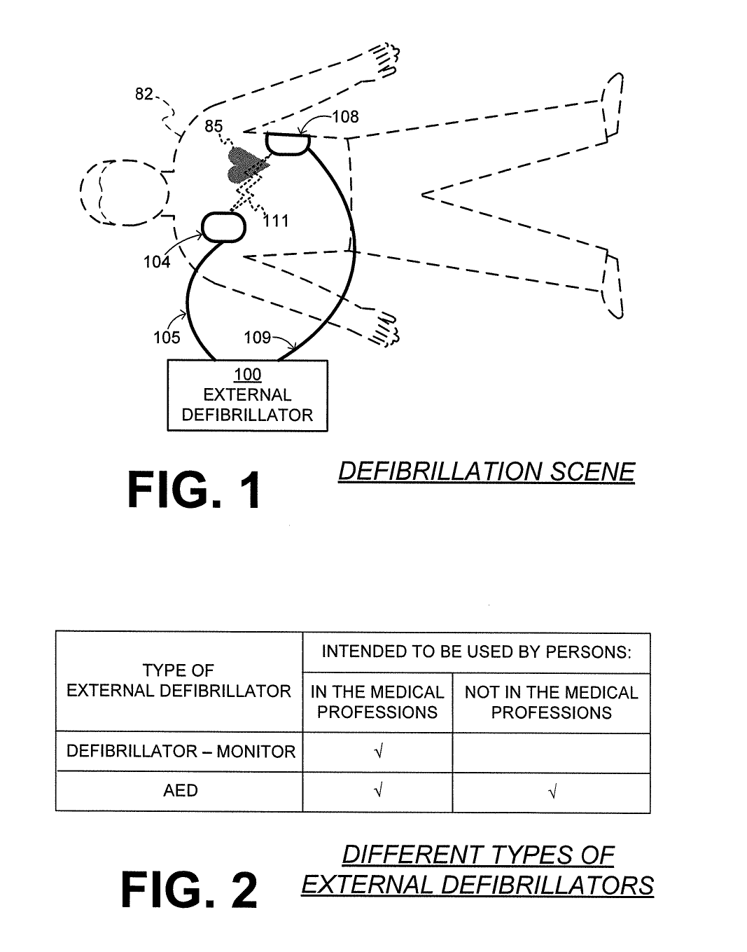

FIG. 1 is a diagram of a defibrillation scene. A person 82 is lying on their back. Person 82 could be a patient in a hospital, or someone found unconscious, and then turned to be on their back. Person 82 is experiencing a condition in their heart 85, which could be Ventricular Fibrillation (VF).

A portable external defibrillator 100 has been brought close to person 82. At least two defibrillation electrodes 104, 108 are usually provided with external defibrillator 100, and are sometimes called electrodes 104, 108. Electrodes 104, 108 are coupled with external defibrillator 100 via respective electrode leads 105, 109. A rescuer (not shown) has attached electrodes 104, 108 to the skin of person 82. Defibrillator 100 is administering, via electrodes 104, 108, a brief, strong electric pulse 111 through the body of person 82. Pulse 111, also known as a defibrillation shock, goes also through heart 85, in an attempt to restart it, for saving the life of person 82.

Defibrillator 100 can be one of different types, each with different sets of features and capabilities. The set of capabilities of defibrillator 100 is determined by planning who would use it, and what training they would be likely to have. Examples are now described.

FIG. 2 is a table listing different types of external defibrillators, and who they are primarily intended to be used by. A first type of defibrillator 100 is generally called a defibrillator-monitor, because it is typically formed as a single unit in combination with a patient monitor. A defibrillator-monitor is sometimes called monitor-defibrillator. A defibrillator-monitor is intended to be used by persons in the medical professions, such as doctors, nurses, paramedics, emergency medical technicians, etc. Such a defibrillator-monitor is intended to be used in a pre-hospital or hospital scenario.

As a defibrillator, the device can be one of different varieties, or even versatile enough to be able to switch among different modes that individually correspond to the varieties. One variety is that of an automated defibrillator, which can determine whether a shock is needed and, if so, charge to a predetermined energy level and instruct the user to administer the shock. Another variety is that of a manual defibrillator, where the user determines the need and controls administering the shock.

As a patient monitor, the device has features additional to what is minimally needed for mere operation as a defibrillator. These features can be for monitoring physiological indicators of a person in an emergency scenario. These physiological indicators are typically monitored as signals. For example, these signals can include a person's full ECG (electrocardiogram) signals, or impedance between two electrodes. Additionally, these signals can be about the person's temperature, non-invasive blood pressure (NIBP), arterial oxygen saturation/pulse oximetry (SpO2), the concentration or partial pressure of carbon dioxide in the respiratory gases, which is also known as capnography, and so on. These signals can be further stored and/or transmitted as patient data.

A second type of external defibrillator 100 is generally called an AED, which stands for “Automated External Defibrillator”. An AED typically makes the shock/no shock determination by itself, automatically. Indeed, it can sense enough physiological conditions of the person 82 via only the shown defibrillation electrodes 104, 108 of FIG. 1. In its present embodiments, an AED can either administer the shock automatically, or instruct the user to do so, e.g. by pushing a button. Being of a much simpler construction, an AED typically costs much less than a defibrillator-monitor. As such, it makes sense for a hospital, for example, to deploy AEDs at its various floors, in case the more expensive defibrillator-monitor is more critically being deployed at an Intensive Care Unit, and so on.

AEDs, however, can also be used by people who are not in the medical profession. More particularly, an AED can be used by many professional first responders, such as policemen, firemen, etc. Even a person with only first-aid training can use one. And AEDs increasingly can supply instructions to whoever is using them.

AEDs are thus particularly useful, because it is so critical to respond quickly, when a person suffers from VF. Indeed, the people who will first reach the VF sufferer may not be in the medical professions.

Increasing awareness has resulted in AEDs being deployed in public or semi-public spaces, so that even a member of the public can use one, if they have obtained first aid and CPR/AED training on their own initiative. This way, defibrillation can be administered soon enough after the onset of VF, to hopefully be effective in rescuing the person.

There are additional types of external defibrillators, which are not listed in FIG. 2. For example, a hybrid defibrillator can have aspects of an AED, and also of a defibrillator-monitor. A usual such aspect is additional ECG monitoring capability.

For patients who qualify for the invasive surgical procedure and an ICD, there is a wait time from the point of diagnosis to the point of the surgical placement of an ICD in a patient. If not monitored, the wait period renders the patient vulnerable to life threatening cardiac episodes. For patients who are vulnerable to cardiac episodes yet are not good candidates for surgery, however, another type of solution, such as by way of an example a wearable defibrillator/monitor, would be highly desirable.

Thus, there is a pressing need for a system, device, method for an automated, continual, and relative to an ICD, non-invasive monitoring and, upon need, immediate therapy administration to a cardiac event victim. As such, a pressing need exists for an improved approach to collecting, storing, transferring to a remote location/medical professional, and analyzing data to ensure quick and accurate medical response to an emergency situation.

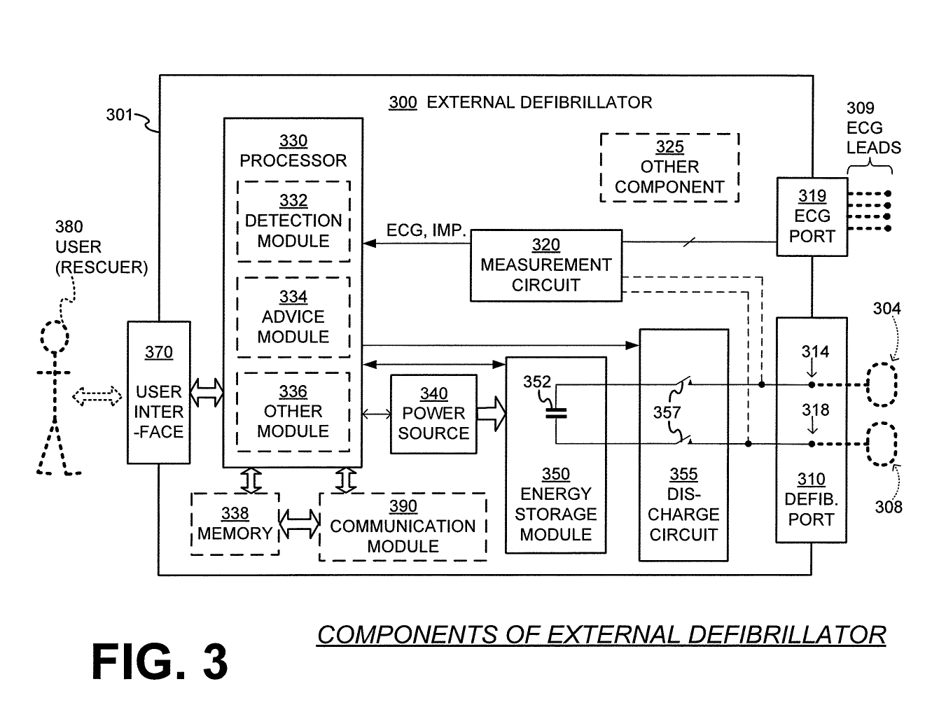

FIG. 3 is a diagram showing components of an external defibrillator 300 made according to embodiments. These components can be, for example, in external defibrillator 100 of FIG. 1. Plus, these components of FIG. 3 can be provided in a housing 301, which is also known as casing 301.

External defibrillator 300 is intended for use by a user 380, who would be the rescuer, or the person 82. Defibrillator 300 typically includes a defibrillation port 310, such as a socket in housing 301. Defibrillation port 310 includes nodes 314, 318. Defibrillation electrodes 304, 308, which can be similar to electrodes 104, 108, can be plugged in defibrillation port 310, so as to make electrical contact with nodes 314, 318, respectively. It is also possible that electrodes can be connected continuously to defibrillation port 310, etc. Either way, defibrillation port 310 can be used for guiding via electrodes to person 82 an electrical charge that has been stored in defibrillator 300, as will be described later in this document.

If defibrillator 300 is actually a defibrillator-monitor, as was described with reference to FIG. 2, then it will typically also have an ECG port 319 in housing 301, for plugging in ECG leads 309. ECG leads 309 can help sense an ECG signal, e.g. a 12-lead signal, or from a different number of leads. Moreover, a defibrillator-monitor could have additional ports (not shown), and an other component 325 for the above described additional features, such as patient signals.

Defibrillator 300 also includes a measurement circuit 320. Measurement circuit 320 receives physiological signals from ECG port 319, and also from other ports, if provided. These physiological signals are sensed, and information about them is rendered by circuit 320 as data, or other signals, etc.

If defibrillator 300 is actually an AED, it may lack ECG port 319. Measurement circuit 320 can obtain physiological signals through nodes 314, 318 instead, when defibrillation electrodes 304, 308 are attached to person 82. In these cases, a person's ECG signal can be sensed as a voltage difference between electrodes 304, 308. Plus, impedance between electrodes 304, 308 can be sensed for detecting, among other things, whether these electrodes 304, 308 have been inadvertently disconnected from the person.

Defibrillator 300 also includes a processor 330. Processor 330 may be implemented in any number of ways. Such ways include, by way of example and not of limitation, digital and/or analog processors such as microprocessors and digital-signal processors (DSPs); controllers such as microcontrollers; software running in a machine; programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

Processor 330 can be considered to have a number of modules. One such module can be a detection module 332, which senses outputs of measurement circuit 320. Detection module 332 can include a VF detector. Thus, the person's sensed ECG can be used to determine whether the person is experiencing VF.

Another such module in processor 330 can be an advice module 334, which arrives at advice based on outputs of detection module 332. Advice module 334 can include a Shock Advisory Algorithm, implement decision rules, and so on. The advice can be to shock, to not shock, to administer other forms of therapy, and so on. If the advice is to shock, some external defibrillator embodiments merely report that to the user, and prompt them to do it. Other embodiments further execute the advice, by administering the shock. If the advice is to administer CPR, defibrillator 300 may further issue prompts for it, and so on.

Processor 330 can include additional modules, such as module 336, for other functions. In addition, if other component 325 is indeed provided, it may be operated in part by processor 330, etc.

Defibrillator 300 optionally further includes a memory 338, which can work together with processor 330. Memory 338 may be implemented in any number of ways. Such ways include, by way of example and not of limitation, nonvolatile memories (NVM), read-only memories (ROM), random access memories (RAM), any combination of these, and so on. Memory 338, if provided, can include programs for processor 330, and so on. The programs can be operational for the inherent needs of processor 330, and can also include protocols and ways that decisions can be made by advice module 334. In addition, memory 338 can store prompts for user 380, etc. Moreover, memory 338 can store patient data.

Defibrillator 300 may also include a power source 340. To enable portability of defibrillator 300, power source 340 typically includes a battery. Such a battery is typically implemented as a battery pack, which can be rechargeable or not. In certain embodiments, a combination is used, of rechargeable and non-rechargeable battery packs. Other embodiments of power source 340 can include AC power override, for where AC power will be available, and so on. In some embodiments, power source 340 is controlled by processor 330.

Defibrillator 300 additionally includes an energy storage module 350. Module 350 is where some electrical energy is stored, when preparing it for sudden discharge to administer a shock. Module 350 can be charged from power source 340 to the right amount of energy, as controlled by processor 330. In typical implementations, module 350 includes one or more capacitors 352, and so on.

Defibrillator 300 moreover includes a discharge circuit 355. Circuit 355 can be controlled to permit the energy stored in module 350 to be discharged to nodes 314, 318, and thus also to defibrillation electrodes 304, 308. Circuit 355 can include one or more switches 357. Those can be made in a number of ways, such as by an H-bridge, and so on.

Defibrillator 300 further includes a user interface 370 for user 380. User interface 370 can be made in any number of ways. For example, interface 370 may include a screen, to display what is detected and measured, provide visual feedback to the rescuer for their resuscitation attempts, and so on. Interface 370 may also include a speaker, to issue voice prompts, etc. Interface 370 may additionally include various controls, such as pushbuttons, keyboards, and so on. In addition, discharge circuit 355 can be controlled by processor 330, or directly by user 380 via user interface 370, and so on.

Defibrillator 300 can optionally include other components. For example, a communication module 390 may be provided for communicating with other machines. Such communication can be performed wirelessly, or via wire, or by infrared communication, and so on. This way, data can be communicated, such as patient data, device information, incident information, therapy attempted, CPR performance, and so on.

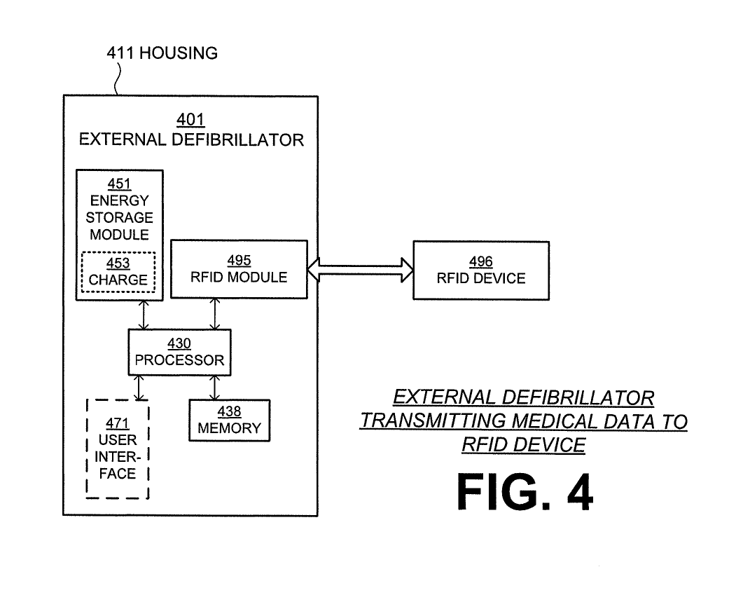

FIG. 4 is a diagram showing an external defibrillator 401 transmitting medical data to an RFID device 496 according to embodiments. As used herein, the terms medial data and medically relevant data generally refer to information pertaining to ECG data, patient activity, wellness information, quality of life information, device-related information, battery status, logs, error, malfunctions, service needs, battery condition, and so on.

In the example, the external defibrillator 401 includes a housing 411, a medical data collection port (not shown) for collecting medical data corresponding to a patient, such as the person using the external defibrillator 401, and a radio frequency identification (RFID) communication module 495 in the housing 411. While the illustrated embodiment is directed to an external defibrillator 401, such as an AED, it will be appreciated that the teachings described herein may be applied to other external medical devices, such as a medical monitoring device or system configured to monitor the patient's cardiac rhythm, for example.

The external defibrillator 401 also includes a processor 430 configured to cause the RFID communication module 495 to transmit the medical data to the RFID device 496, which is external to the external defibrillator 401. The RFID device 496 may be an RFID card, an RFID tag, or an RFID reader, for example. If an RFID reader, the device 496 may include a user interface, a memory, or both, for example.

In certain embodiments, the external defibrillator 401 also includes a memory 438 adapted to store the medical data internally. In alternative embodiments, the memory 438 may include a removable memory device, such as a smart memory card or thumb drive.

The external defibrillator 401 may optionally include a user interface 471 to facilitate interactions with a user, such as the person using the external defibrillator 401. For example, the processor 430 may cause the RFID module 495 to transmit the medical data to the RFID device 496 responsive to a user request received by the user interface 471.

The external defibrillator 401 may also include an energy storage module 451 in an interior of the housing 411 for storing an electrical charge 453, a defibrillation port (not shown) for guiding via electrodes the electrical charge 453 to a person, such as the person using the external defibrillator 401.

In certain embodiments, the external defibrillator 401 may be configured to be worn by a user, such as the person using the external defibrillator 401.

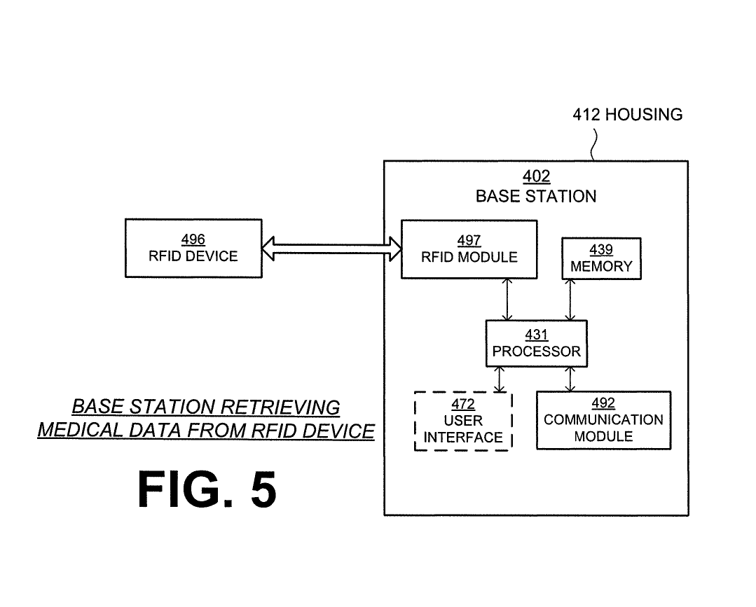

FIG. 5 is a diagram showing the RFID device 496 of FIG. 4 providing at least some of the medical data received from the external defibrillator 401 to a base station 402 according to embodiments. The RFID device 496 may be an RFID card, and RFID tag, or any of a number of other suitable RFID devices. In certain embodiments, the RFID device 496 may be situated within or otherwise integrated with the base station 402.

The base station 402 includes a housing 412 and an RFID communication module 497 in the housing 412. The RFID communication module 497 may include an RFID reader, for example. The base station 402 also includes a processor 431 configured to cause the RFID communication module 497 to retrieve or otherwise receive the medical data from the RFID device 496. In certain embodiments, the medical data may be erased from the RFID device 496 after successful receipt of the medical data by the RFID communication module 497.

In certain embodiments, the base station 402 also includes a memory 439 adapted to store the medical data internally. The memory 439 may be any of a number of suitable memory devices. In such embodiments, the memory 439 may be removable from the base station 402.

The base station 402 may optionally include a user interface 472 to facilitate interactions with a user, such as the person using the external defibrillator 401. For example, the processor 431 may cause the RFID module 496 to retrieve or otherwise receive the medical data from the RFID device 496 responsive to a user request received by the user interface 472.

The base station 402 may include a communication module 492 configured to establish communication with one or more external networks, such as the Internet. The communication module 492 may transmit or otherwise provide at least some of the medical data to a remote or central database or service, for example.

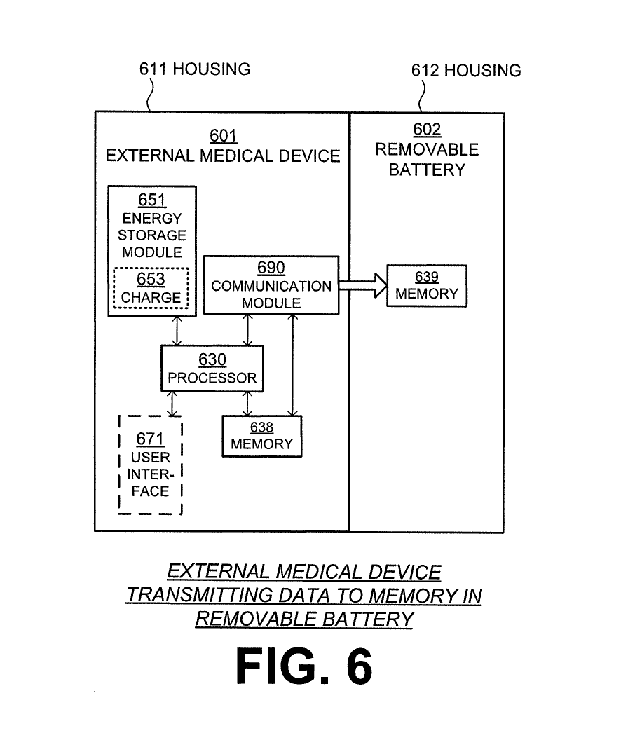

FIG. 6 is a diagram showing an external medical device 601 transmitting medical data to a memory device 639 in a removable battery 602 according to embodiments. The external medical device 601 includes a housing 611, a medical data collection port (not shown) for collecting medical data corresponding to a person using the external medical device 601, and a communication module 690 in the housing 611. The external medical device 601 may be any of a number of different external medical devices, such as a medical monitoring device or system configured to monitor the patient's cardiac rhythm, for example. Such devices may each be configured to be wearable by a person such as the user thereof, for example.

The removable battery 602 includes a housing 612. In certain embodiments, the memory device 639 is positioned entirely within the housing 612. In other embodiments, the memory device 639 is at least partially outside the housing 612 but integrated with or otherwise physically coupled to the housing 612. The memory device 639 may be a flash memory device, for example. In certain embodiments, the memory device 639 may be removable from the battery 602. In such embodiments, the memory device 639 may be configured to only receive the medical data from the communication module 690 while the removable battery 602 is coupled with the external medical device 601.

In certain embodiments, the removable battery 602 may include an RFID device suitable for receiving the medical data from the external medical device 601, for example. In such embodiments, the RFID device may be in place of, or in addition to, the memory 639.

The external medical device 601 also includes a processor 630 configured to cause the communication module 690 to transmit the medical data to the memory device 639. In certain embodiments, the external medical device 601 also includes a memory 638 adapted to store the medical data internally. In alternative embodiments, the memory 638 may include a removable memory device, such as a smart memory card or thumb drive.

The external medical device 601 may optionally include a user interface 671 to facilitate interactions with a user, such as the person using the external medical device 601. For example, the processor 630 may cause the communication module 690 to transmit the medical data to the memory device 639 responsive to a user request received by the user interface 671.

The external medical device 601 may also include an energy storage module 651 in an interior of the housing 611 for storing an electrical charge 653, a defibrillation port (not shown) for guiding via electrodes the electrical charge 653 to a person, such as the person using the external medical device 601.

In certain embodiments, the external medical device 601 may be configured to be worn by a user, such as the person using the external medical device 601.

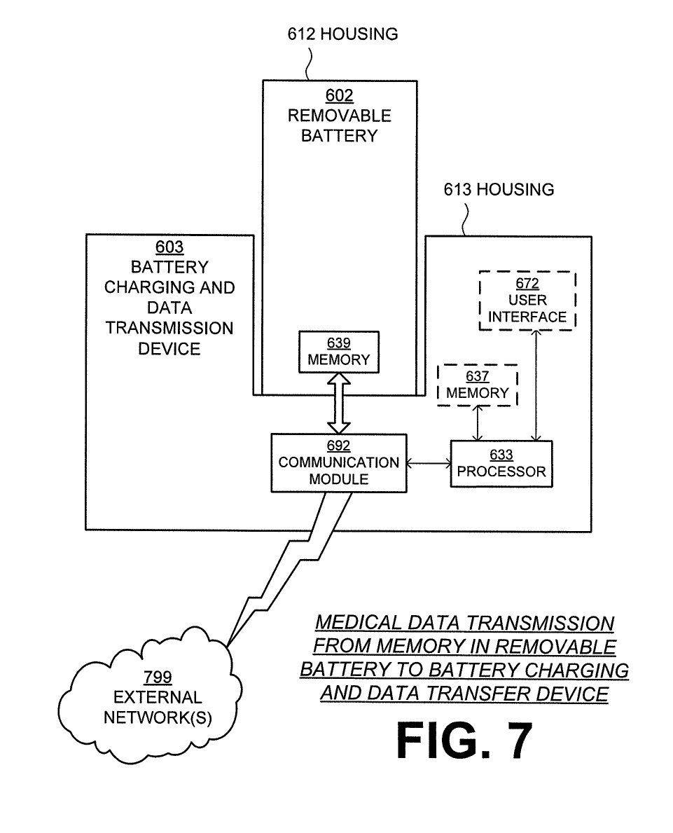

FIG. 7 is a diagram showing the memory device 639 of FIG. 6 providing at least some of the medical data to a battery charging and data transfer device 603 according to embodiments.

The battery charging and data transfer device 603 includes a housing 613 and a communication module 692 in the housing 613. The battery charging and data transfer device 603 also includes a processor 633 configured to cause the communication module 692 to retrieve or otherwise receive the medical data from the memory device 639 integrated with the removable battery 602. The connection between the memory 639 and the communication module 692 may be physical, wireless, or of some other suitable type of connection.

In certain embodiments, the medical data may be erased from the memory device 639, e.g., after successful transmission of the medical data to the communication module 692.

In certain embodiments, the battery charging and data transfer device 603 may be configured to receive the medical data from the memory device 639 while the removable battery 602 is coupled with the battery charging and data transfer device 603. Alternatively or in addition thereto, the battery charging and data transfer device 603 may be configured to receive the medical data from the memory device 639 while the battery charging and data transfer device 603 is providing a charge to the removable battery 602.

In certain embodiments, the battery charging and data transfer device 603 also includes a memory 637 adapted to store the medical data internally. The memory 637 may be any of a number of suitable memory devices. In such embodiments, the memory 637 may be removable from the battery charging and data transfer device 603.

The battery charging and data transfer device 603 may optionally include a user interface 672 to facilitate interactions with a user, such as the person using the external medical device 601. For example, the processor 633 may cause the communication module 692 to retrieve or otherwise receive the medical data from the memory device 639 responsive to a user request received by the user interface 672.

The communication module 692 may be further configured to establish communication with one or more external networks 799, such as the Internet. The communication module 692 may transmit or otherwise provide at least some of the medical data to a remote or central database or service, for example.

The functions of this description may be implemented by one or more devices that include logic circuitry. The device performs functions and/or methods as are described in this document. The logic circuitry may include a processor that may be programmable for a general purpose, or dedicated, such as microcontroller, a microprocessor, a Digital Signal Processor (DSP), etc. For example, the device may be a digital computer like device, such as a general-purpose computer selectively activated or reconfigured by a computer program stored in the computer. Alternately, the device may be implemented by an Application Specific Integrated Circuit (ASIC), etc.

Moreover, methods are described below. The methods and algorithms presented herein are not necessarily inherently associated with any particular computer or other apparatus. Rather, various general-purpose machines may be used with programs in accordance with the teachings herein, or it may prove more convenient to construct more specialized apparatus to perform the required method steps. The required structure for a variety of these machines will become apparent from this description.

In all cases there should be borne in mind the distinction between methods in this description, and the method of operating a computing machine. This description relates both to methods in general, and also to steps for operating a computer and for processing electrical or other physical signals to generate other desired physical signals.

Programs are additionally included in this description, as are methods of operation of the programs. A program is generally defined as a group of steps leading to a desired result, due to their nature and their sequence. A program is usually advantageously implemented as a program for a computing machine, such as a general-purpose computer, a special purpose computer, a microprocessor, etc.

Storage media are additionally included in this description. Such media, individually or in combination with others, have stored thereon instructions of a program made according to certain embodiments. A storage medium according to certain embodiments is a computer-readable medium, such as a memory, and is read by the computing machine mentioned above.

Performing the steps or instructions of a program requires physical manipulations of physical quantities. Usually, though not necessarily, these quantities may be transferred, combined, compared, and otherwise manipulated or processed according to the instructions, and they may also be stored in a computer-readable medium. These quantities include, for example electrical, magnetic, and electromagnetic signals, and also states of matter that can be queried by such signals. It is convenient at times, principally for reasons of common usage, to refer to these quantities as bits, data bits, samples, values, symbols, characters, images, terms, numbers, or the like. It should be borne in mind, however, that all of these and similar terms are associated with the appropriate physical quantities, and that these terms are merely convenient labels applied to these physical quantities, individually or in groups.

This detailed description is presented largely in terms of flowcharts, display images, algorithms, and symbolic representations of operations of data bits within at least one computer readable medium, such as a memory. Indeed, such descriptions and representations are the type of convenient labels used by those skilled in programming and/or the data processing arts to effectively convey the substance of their work to others skilled in the art. A person skilled in the art of programming may use these descriptions to readily generate specific instructions for implementing a program according to certain embodiments of the disclosed technology.

Often, for the sake of convenience, it is preferred to implement and describe a program as various interconnected distinct software modules or features, individually and collectively also known as software. This is not necessary, however, and there may be cases where modules are equivalently aggregated into a single program with unclear boundaries. In any event, the software modules or features of this description may be implemented by themselves, or in combination with others. Even though it is said that the program may be stored in a computer-readable medium, in view of the present disclosure, it should be clear to a person skilled in the art that it need not be a single memory, or even a single machine. Various portions, modules or features of it may reside in separate memories, or even separate machines. The separate machines may be connected directly, or through a network, such as a local access network (LAN), or a global network, such as the Internet.

In view of the present disclosure, it will be appreciated that some of these methods may include software steps that may be performed by different modules of an overall software architecture. For example, data forwarding in a router may be performed in a data plane, which consults a local routing table. Collection of performance data may also be performed in a data plane. The performance data may be processed in a control plane, which accordingly may update the local routing table, in addition to neighboring ones. In view of the present disclosure, a person skilled in the art will discern which step is best performed in which plane.

An economy is achieved in the present document in that a single set of flowcharts is used to describe both programs, and also methods. So, while flowcharts are described in terms of boxes, they can mean both method and programs.

For this description, the methods may be implemented by machine operations. In other words, embodiments of programs are made such that they perform methods that are described in this document. These may be optionally performed in conjunction with one or more human operators performing some, but not all of them. As per the above, the users need not be collocated with each other, but each only with a machine that houses a portion of the program. Alternately, some of these machines may operate automatically, without users and/or independently from each other.

Methods are now described.

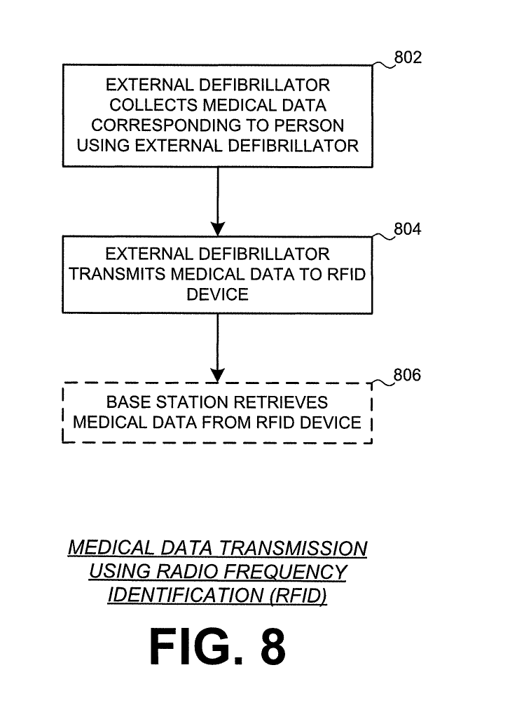

FIG. 8 is a flowchart 800 for illustrating example methods executable by external defibrillators according to embodiments.

In an operation at 802, an external defibrillator—or other suitable device, such as a person monitor—collects medical data corresponding to a person using the external defibrillator. The external defibrillator may be configured to be worn by a person. In such embodiments, the person using the external defibrillator may be wearing the external defibrillator during the medical data collection thereby.

In an operation at 804, a radio frequency identification (RFID) module of the external defibrillator transmits or otherwise provides at least some of the medical data collected at 802 to an external RFID device, such as an RFID tag or card.

In an optional operation at 806, the RFID device may provide at least some of the medical data to a base station. The base station may request the medical data responsive to a user request received from a user interface of the base station, for example.

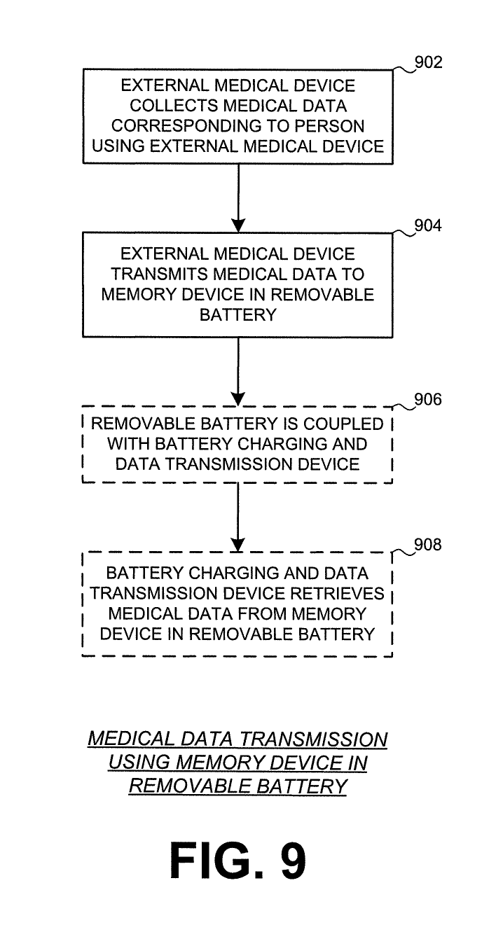

FIG. 9 is a flowchart 900 for illustrating example methods executable by external medical devices according to embodiments.

In an operation at 902, an external medical device collects medical data corresponding to a person using the external medical device. The external medical device may be configured to be worn by a person, in part or in whole. In such embodiments, the person using the external medical device may be wearing the external medical device during the medical data collection thereby.

In an operation at 904, a communication module of the external medical device transmits the medical data to a memory device integrated with a removable battery. The memory device may be positioned within, integrated with, or otherwise physically coupled with the removable battery. In certain embodiments, the memory device may be removable from the battery. The memory device may be configured to only receive the medical data from the communication module while the removable battery is in communication with, in electrical connection with, or otherwise coupled with the external medical device.

In an optional operation at 906, the memory device provides at least some of the medical data to a base station. The base station may include a battery charging station, for example. In such embodiments, the base station may be configured to receive the medical data from the memory device while the removable battery is in communication with, in electrical connection with, or otherwise coupled with the base station.

In an optional operation at 908, the base station transmits or otherwise provides at least some of the medical data received from the memory device to at least one remote service by way of a network, such as the Internet. In certain embodiments, the base station may receive various types of information such as device settings, upgrades, patient instructions, or any combination thereof from a remote service through the network, for example. The base station may transfer any or all of the received information to the memory device such that, when the battery is subsequently coupled with the external medical device, the memory device may provide any or all of the information to the external medical device. Alternatively, such information may be stored for later use.

In this description, numerous details have been set forth in order to provide a thorough understanding. In other instances, well-known features have not been described in detail in order to not obscure unnecessarily the description.

A person skilled in the art will be able to practice embodiments of the disclosed technology in view of the present description, which is to be taken as a whole. The specific embodiments as disclosed and illustrated herein are not to be considered in a limiting sense. Indeed, it should be readily apparent to those skilled in the art that what is described herein may be modified in numerous ways. Such ways can include equivalents to what is described herein. In addition, certain embodiments may be practiced in combination with other systems.

Other embodiments may include combinations and sub-combinations of features described herein including for example, embodiments that are equivalent to providing or applying a feature in a different order than in a described embodiment, extracting an individual feature from one embodiment and inserting such feature into another embodiment, removing one or more features from an embodiment, or both removing a feature from an embodiment and adding a feature extracted from another embodiment, while providing the advantages of such features incorporated in such combinations and sub-combinations.

What is claimed is:

1. An external medical device, comprising:
a medical data collection port configured to collect medical data corresponding to a patient;
a radio frequency identification (RFID) communication module; and
a processor configured to cause the RFID communication module to transmit the medical data to an RFID device that is external to the external medical device.

2. The external medical device of claim 1, wherein the RFID device is an RFID tag.

3. The external medical device of claim 1, wherein the RFID device is an RFID card.

4. The external medical device of claim 1, wherein the RFID device is configured to provide at least some of the medical data to a base station that is external to the external medical device.

5. The external medical device of claim 4, wherein the base station includes an RFID reader.

6. The external medical device of claim 4, wherein the RFID device is configured to provide the medical data to the base station responsive to the base station requesting the medical data from the RFID device.

7. The external medical device of claim 6, wherein the base station includes a user interface, and wherein the base station requests the medical data responsive to a user request received by the user interface.

8. The external medical device of claim 4, wherein the base station includes a user interface, and wherein the processor is configured to cause the RFID module to transmit the medical data to the base station responsive to a user request received by the user interface.

9. The external medical device of claim 1, wherein the external medical device is configured to be worn.

10. The external medical device of claim 9, wherein the external medical device is worn by the patient.

11. The external defibrillator of claim 1, further comprising:
a user interface, and in which
the processor is configured to cause the RFID module to transmit the medical data to the RFID device responsive to a user request received by the user interface.

12. The external medical device of claim 1, further comprising:
an energy storage module configured to store an electrical charge; and
a defibrillation port configured to deliver the electrical charge to the patient.

13. The external medical device of claim 1, wherein the patient is a person using the external medical device.

14. A system, comprising:
a radio frequency identification (RFID) device; and
an external defibrillator configured to collect medical data corresponding to a patient, the external defibrillator including a radio frequency identification (RFID) module configured to transmit the medical data to the RFID device.

15. The system of claim 14, further comprising:
a base station that is external to the external defibrillator and configured to receive at least some of the medical data from the RFID device.

16. The system of claim 14, wherein the patient is a person using the external defibrillator.

17. The system of claim 16, wherein the person using the external defibrillator is wearing the external defibrillator.

18. A method, comprising:
an external defibrillator collecting medical data corresponding to a person using the external defibrillator; and
a radio frequency identification (RFID) module of the external defibrillator transmitting the medical data to an RFID device that is external to the external defibrillator.

19. The method of claim 18, further comprising:
the RFID device providing at least some of the medical data to a base station that is external to the external defibrillator.

20. The method of claim 18, further comprising:
erasing the medical data from the RFID device.
